(19) United States
(12) Patent Application Publication
(10) Pub. No.: US 2009/0264978 A1
(43) Pub. Date: Oct. 22, 2009

(54) Friction-Release Distal Latch Implant Delivery System and Components

(76) Inventors: Martin S. Dieck, Campbell, CA (US); Frank P. Becking, Santa Clara, CA (US); Maria G. Aboytes, Palo Alto, CA (US)

Correspondence Address: ORRICK, HERRINGTON & SUTCLIFFE, LLP;IP PROSECUTION DEPARTMENT, 4 PARK PLAZA, SUITE 1600, IRVINE, CA, 92614-2558, US

(21) Appl. No.: 12/412,731

(22) Filed: Mar. 27, 2009

Related U.S. Application Data
(60) Provisional application No. 61/039,863, filed on Mar. 27, 2008.
(60) Provisional application No. 61/158,456, filed on Mar. 9, 2009.

(51) Int. Cl. A61F 2/06

(52) U.S. Cl. 623/1.11; 623/1.23

(57) ABSTRACT

Provided herein are systems, devices and methods for the delivery of medical implants. A distal end portion of the implant is coupled with a delivery device by surface friction between the implant and an underlying surface such that the distal end portion is frictionally locked and maintained in the appropriate position and state prior to delivery. When positioned within the patient at the proper location, the state of frictional lock can be released to free the distal end portion of the implant from the delivery device.

### CROSS-REFERENCE TO RELATED APPLICATIONS

This application claims the benefit of U.S. Provisional Application Ser. Nos. 61/039,863, filed Mar. 27, 2008, and 61/158,456, filed Mar. 9, 2009, each of which is hereby fully incorporated by reference.

### FIELD OF THE INVENTION

The subject matter described herein relates generally to systems, devices and methods for the delivery of textured (e.g., braided or woven) medical implants.

### BACKGROUND OF THE INVENTION

US Patent Publications 2006/0271149 and 2006/0271153, assigned to CHESTNUT MEDICAL TECHNOLOGIES, INC., disclose delivery systems for braid-type stents. In one example system, a distal coil socket holds the distal end of the braid stent until the braid is retracted by grippers holding the proximal end. These grippers are able to maintain contact with the proximal end through compression by an external sleeve surrounding the grippers. Upon sleeve withdrawal, the grippers release the proximal end of the stent.

System miniaturization of the referenced system(s) is limited by the gripper configuration. Also, the lack of a release mechanism for detachment from the distal socket presents issues of inadvertent deployment and/or non-optimal control. Accordingly, there remains a need for both more robust/reliable and potentially further downsizable systems for advanced braid-type implant delivery. The present invention offers such systems with various advantages as presented herein and others as may be apparent to those with skill in the art.

### SUMMARY

The systems, methods and devices described in this section and elsewhere herein are done so by way of example embodiments. These example embodiments are provided to aid in the description of the inventive subject matter and are in no way intended to limit the inventive subject matter beyond the express language of the claims. For example, the inventive subject matter described herein is directed towards implant securement through releasable surface friction generated between a textured implant and a textured delivery device, example embodiments of which are braided implants and multi-filar or braided delivery devices. However, this inventive subject matter is not limited solely to the use of braided or multi-filar configurations as one of skill in the art will appreciate, based on this disclosure, that other textured configurations can likewise provide satisfactory surface friction. Thus, the embodiments provided herein for this and all other features are merely non-exhaustive examples.

Provided herein are systems, devices and methods for implant delivery with a device that holds the implant in a state of frictional lock. This application claims the benefit of U.S. Provisional Application Ser. Nos. 61/039,863, filed Mar. 27, 2008 and 61/158,456, filed Mar. 9, 2009, each of which is hereby fully incorporated by reference. The implant is preferably (i.e., has been selected as but is not necessarily) a stent and its distal end portion is held onto a core construct in a state of frictional lock by a distal housing (or latch). A proximal housing or other holding or grasping device can be used to retain the proximal end portion of the implant in a state of frictional lock or otherwise. The core construct can comprise an elongate tubular textured member, e.g., a braided or multi-filar sleeve, slidable over an elongate core member (or central wire). The sleeve preferably includes at least an accessible (or exposed) distal textured interface for contact with a corresponding textured surface on the implant. The sleeve can also include an optional proximal textured interface for contact with a corresponding textured surface on the implant. These interfaces are preferably present about the periphery of the sleeve, but can also be limited to smaller regions, with the distal implant interface being adjacent the distal end of the sleeve. In a preferred example embodiment, the sleeve is a braided tube that is covered (or jacketed) between the implant interface regions. The covering is preferably fixed to the braid and can be formed from a heat-shrinkable tube, extrusion, and the like.

Alternatively, or additionally, a proximal portion of the braid may comprise a secondary jacket to stiffen it relative to one or more distal and more flexible sections. Such a construction for the sleeve is highly pushable, torqueable and kink-resistant. Moreover, in a braided configuration, the sleeve can have its PIC (Per Inch Crosses) varied along its length to provide enhanced distal flexibility. In other words, the sleeve may be tuned/modified as a catheter-like subcomponent of the system. In an alternative embodiment, an elongate polymeric, metallic or metal alloy shaft can be used with sections of braid attached (e.g., clamped, glued, embedded or the like) to the shaft surface to form the interfaces with the implant.

Similarly, the core member can also be configured for enhanced flexibility. For example, the core member may have one or more successively tapered regions near or adjacent to its distal end, like a typical guidewire. The core member is preferably coupled with an atraumatic distal end (e.g., a floppy coil tip). Both the core member and the sleeve can comprise an elastic or superelastic materials such as stainless steel, NiTi, CoCr, other alloys, polymeric materials and the like.

The tubular implant preferably has textured distal and proximal surfaces (which may be continuous or disconnected). These surfaces are preferably present about the entire inner periphery of the implant, but can also be located in limited regions generally corresponding to the interface regions of the sleeve. In a preferred embodiment, the implant is a braided implant with a braided surface about its entire exterior. However, other configurations of implants having grafts, coatings (e.g., lubricious, drug-eluting, and the like) or other non-textured surfaces present on the exterior of the implant are possible. See, e.g., U.S. Pat. No. 4,416,028 to Eriksson, et al.

The tubular implant is expandable from a contracted state to an expanded state, and preferably self-biased towards the expanded state. Generally, expansion results in lengthwise shortening of the implant. Thus, holding the end portions of the implant stretched apart from each other (such as in the state of frictional lock described herein) can cause the implant to be maintained in a contracted state, without the need to radially restrain the entire implant (such as with a full body sheath). If the implant is self-biased to expand, release of the end portions allows the implant to expand into apposition with tissue at the implantation site. Else, a secondary expansion device can be used, such as an inflatable balloon or mechanical arms.

The frictional lock described herein relies on a high degree of surface friction between the implant and an underlying surface to resist longitudinal/axial motion of the implant (in its contracted state) along the longitudinal axis of the delivery device or sleeve. Substantial surface friction between implant and the underlying surface will prevent the implant from sliding relative to the underlying surface, preventing the implant from decreasing in length (i.e., for shortening) and radially expanding.

Although the term “lock” can be used, it should be understood that the implant is not locked from all movement in an absolute sense, as the implant can be forced from the lock should sufficient force be applied to overcome the surface friction. Rather, the implant is preferably locked in place sufficiently to resist the implant's own bias towards expansion (if any), to resist bias applied by a secondary expansion device (if any), to resist forces applied against the implant while maneuvering within the patient's vasculature (e.g., forces applied either by the delivery device or the patient's vasculature or blood flow), and/or to resist forces applied to the implant during any loading, unloading, or deployment procedures. Of course, one of skill in the art will appreciate that the degree of surface friction necessary to achieve the state of frictional lock will depend on the specific delivery device implementation and intended application(s).

It has been found that certain textured surfaces, when in opposition to each other, are capable of exhibiting sufficient surface friction to form a frictional lock for implant delivery. The term “textured” is not intended to imply the use of any particular material or manufacturing process (e.g., an applied finish or coating). Instead, the term “textured” is used in a basic sense only to refer to surface profile, namely, a non-level or high-friction surface profile, as opposed to a level, smooth or polished surface profile. Certain of these textured surfaces can be formed from many smaller, discrete constituents in close proximity with each other, such as with braids, meshes, matrices and fabrics, which are generally formed from one or more layers of woven or interleaved strands, threads or wires, and multi-filar materials, which are generally formed from windings or coils of strands, threads or wires. Examples of these used to create frictional lock (implant-to-sleeve or sleeve-to-implant) include braid-to-braid contact, multi-filar-to-multi-filar contact, and braid-to-multi-filar contact. The same or similar configurations of the textured material generally generate the greatest surface friction, i.e., two braids having the same number and size of constituents, identical PIC and pitch (the angle of the constituent with respect to an axis of the braid), since the opposing constituents are readily placed in interfering/interlacing contact with each other. These configurations also have the advantage that flexing, twisting or stretching can force the constituents into even greater contact or interference, further increasing the frictional lock. Other textured surfaces can be formed on a body by deforming this surface to create a textured pattern, e.g., by etching, grinding, sanding, and the like. Still other textured surfaces can be formed by applying a high-friction coating to a body. Of course, any combination of these can also be used (e.g., a braid implant on a patterned underlying surface, etc.).

The implant is preferably held engaged with the underlying interfaces by a distal and a proximal housing (or cover) that closely fits over at least the distal and proximal end portions of the implant, respectively, such that the implant is held (or constrained) in contact with or against the respective underlying interfaces of the sleeve. Should it be desired, the core member can abut the sleeve from the interior, to resist inward deformation by the sleeve when the implant is pressed against it by the housings. Here, the implant is frictionally locked when held against the sleeve by the distal and/or proximal housings. It should be noted that the entire end portion of the implant need not be housed by a continuous covering, only so much as to adequately hold the implant end portion in the contracted state and in frictional lock with the underlying surface.

In one example embodiment, at least one of the distal and proximal housings are moveable with respect to the other to release the implant from frictional lock. For example, the distal housing can be fixed to the core member and can slide relative to the sleeve or proximal housing by movement of the core member. Advancement of the distal housing off of the implant releases the distal lock. The proximal housing can be a retractable tubular member placed over the sleeve and can slide relative to the sleeve or distal housing. Retraction of the proximal housing releases the proximal lock.

In another example embodiment, the distal housing can be fixed to the sleeve, with the core member remaining slidable within. The core member preferably includes a distally-located wedge-like portion that holds the sleeve in an open state against the implant from the interior at the distal interface (and also, optionally, the proximal interface). The distal lock can be released by proximally retracting the core member within the sleeve, which allows the sleeve (advantageously heatset or otherwise set to a smaller diameter) to collapse/withdraw from the distal textured portion of the implant and reduce the degree of contact (partially or entirely) with the implant. The proximal lock can be similarly released (in which case it can be fixed to the sleeve) or the proximal housing can optionally be made retractable as described above.

In one example embodiment, the distal and proximal housings are configured as tubular sheaths. These tubular sheaths can, for example, be formed by heat-shrinkable tubing. The heatshrink for the housings, and the jacket described above, may be PE (polyethylene), PET (polyester), or the like. PI (polyamide), FEP, PEEK and other materials may also be advantageously employed. The housings can be formed in sections of the tubular sheaths that have a relatively larger diameter than adjacent sections, e.g., the housing can be a section of the sheath that shoulders outward.

The distal housing can extend between about 0.5 to about 5 mm (millimeter) over the sleeve, effectively serving as a distal mini-sheath (i.e., a sheath that covers less than the entire delivery device). At the proximal housing, the tubing can similarly overlap the braid, and run the length of the delivery system to a handle providing a proximal mini-sheath. In this fashion, the proximal and distal housings are in spaced relation to each other, leaving a central section of the underlying sleeve exposed.

Such an approach allows for a small overall diameter system. The proximal mini-sheath may comprise thinner material than would be required for a full-length sheath because it pulls off the stent more easily with less of the implant covered and need not be as robust as in cases higher withdrawal forces are encountered.

As such, the preferred example braided implant is held closely by the covered sections in a stretched (reduced diameter) configuration. The implant's number of wires, profile, diameter, etc. may range in size. The braid shown in the incorporated provisional applications (61/039,863 & 61/158,456) is a very fine NiTi mesh/matrix available from Secant Medical. The braid may be metallic (as in NiTi, St. Steel, CoCr, etc.), polymeric, of hybrid construction, and the like.

Again, an important aspect of the system is that the engagement between implant and sleeve is robust enough to securely hold the braid in the contracted state (e.g., stretched lengthwise) when captured at both ends. Advantageously, while the surface friction between implant and underlying sleeve interfaces is high, the surface friction between the implant and the over-lying housings (e.g., the mini-sheaths) is much lower, allowing the housing to readily slide over the implant without causing the implant to slide over the underlying sleeve interface, thereby facilitating delivery.

In a preferred example use, the delivery system is inserted into the patient's vasculature and pushed and navigated to a treatment site using conventional techniques just as if it were a guidewire. However, it may simply be passed through a catheter after exchange with a guidewire. Accordingly, for neurovascular applications, the system is advantageously sized to cross either an 0.021 or 0.027 inch microcatheter. The device is feasibly made with as small as about an 0.018 inches diameter. It may still be useful at larger sizes (especially for other applications—such as in the coronary or peripheral vasculature) as well.

After advancement to the treatment site, the implant is delivered by releasing or disengaging the implant from the state of frictional lock, i.e., allowing the textured surface of the implant to transition out of locking contact with the underlying textured surface). It may be advantageous to first release the distal lock in one of the manners described herein, such as by relative movement between the sleeve and distal housing (i.e., by advancing the core/distal housing relative to the sleeve, or withdrawing the sleeve relative to the core/distal housing). When one side is released, the implant partially opens and foreshortens. The physician (or other medical professional) implanting the device may choose to confirm location (e.g., via fluoroscopy), reposition and/or withdraw the device while the braid-stent is still captured at the proximal (or distal) end portion. If placement is satisfactory, the proximal lock can be released in one of the manners described herein, such as by relative movement between the sleeve or core member and proximal sheath.

The implant may be so-delivered for a number of purposes. With a braided stent, at higher densities (e.g., with a stent as pictured in the incorporated provisional applications), it may be used to disrupt/divert the flow to treat an aneurysm or fistula. It may be also be used as a “coil jailer” by first trapping a microcatheter between the stent and a vessel wall and delivering coils into an aneurysm. It may be used as a liner, followed by placement of a tube-cut stent within it when stenting diseased saphenous vein graphs. Other possibilities exist as well or will be apparent to those of ordinary skill in the art. The inventive subject matter provided herein includes these methods, systems and devices for practicing these methods, and methods of manufacturing those systems and devices.

It should be noted that the elongate textured member (or sleeve) can bear universal application to other treatment systems and methods. For instance, the sleeve sub-assembly can be used with a wide array of different implants and locking mechanisms, not limited to braided stents or distal/proximal housings. The elongate textured member sub-assembly is adapted for insertion into the body of a patient in its finished form. It can also be coupled with an actuator located external to the patient at or near its proximal end. It preferably includes implant-accessible textured surfaces, or implant interfaces, located at distal and proximal locations selected corresponding to the implant. As mentioned, these surfaces preferably extend about the outer periphery of the elongate sleeve. A non-textured surface, which also preferably extends about the outer periphery of the braid, is located between the first and second textured surfaces. In one example embodiment, the elongate textured member is a braided tubular member with a covering (e.g., a polymeric jacket) placed, and preferably secured or fixed, overtop. It may be glued, fused or heat-shrink(ed) in place. The non-textured surface is the surface of the covering and the proximal and distal textured surfaces are exposed surfaces of the braid, accessible to the implant. The elongate textured member can also include another covering located proximal to the proximal textured surface. This other covering preferably runs the length of the member to or near the proximal end and lends support to the member, e.g., increasing its pushability.

In the finished form, a braided and covered sub-assembly is preferably ready to be used in the medical procedure. The manufacturing of the braid is preferably complete and any treatment to the braid ends (e.g., heatsetting, welding, potting, etc.) to prevent fraying is also complete. The covering is securely fixed to the braid and has hardened and been otherwise treated.

Other systems, methods, features and advantages of the subject matter described herein will be or will become apparent to one with skill in the art upon examination of the following figures and detailed description. Still further, it includes methods associated with and/or activities implicit to the use of the devices described. It is intended that all such additional systems, methods, features and advantages be included within this description, be within the scope of the subject matter described herein, and be protected by the accompanying claims. In no way should the features of the example embodiments be construed as limiting the appended claims, absent express recitation of those features in the claims.

### BRIEF DESCRIPTION OF THE FIGURES

The details of the inventive subject matter set forth herein, both as to its structure and operation, may be appreciated, in part, by study of the accompanying figures, in which like reference numerals refer to like parts. The components in the figures are not necessarily to scale, emphasis instead being placed upon illustrating the principles of the subject matter. Moreover, all illustrations are intended to convey concepts, where relative sizes, shapes and other detailed attributes may be illustrated schematically rather than literally or precisely. Variation from the embodiments depicted is, of course, contemplated. Moreover, details commonly understood by those with skill in the art may be omitted as will be understood in review of the figures. Of these:

FIGS. 1A-C are side views depicting an example embodiment of the implant delivery system at different stages of implant deployment; FIG. 2A is a side view depicting another example embodiment of the implant delivery system;

FIG. 2B is an assembly view depicting components of the example embodiment of the implant delivery system of FIG. 2A; FIGS. 2C-D are side views depicting the example embodiment of FIGS. 2A-B at different stages of implant deployment; and FIGS. 3A-B are side views depicting another example embodiment of the implant delivery system at different stages of implant deployment.

In these views, elements that are contained within other elements are shown in profile with broken lines. However, though sometimes partially obscured, the implant profile is illustrated using an “x x x x x” pattern.

### DETAILED DESCRIPTION

Provided herein are systems, devices and methods for the delivery of a preferably expandable implant using one or more devices for releasably holding the implant in a state of frictional lock.

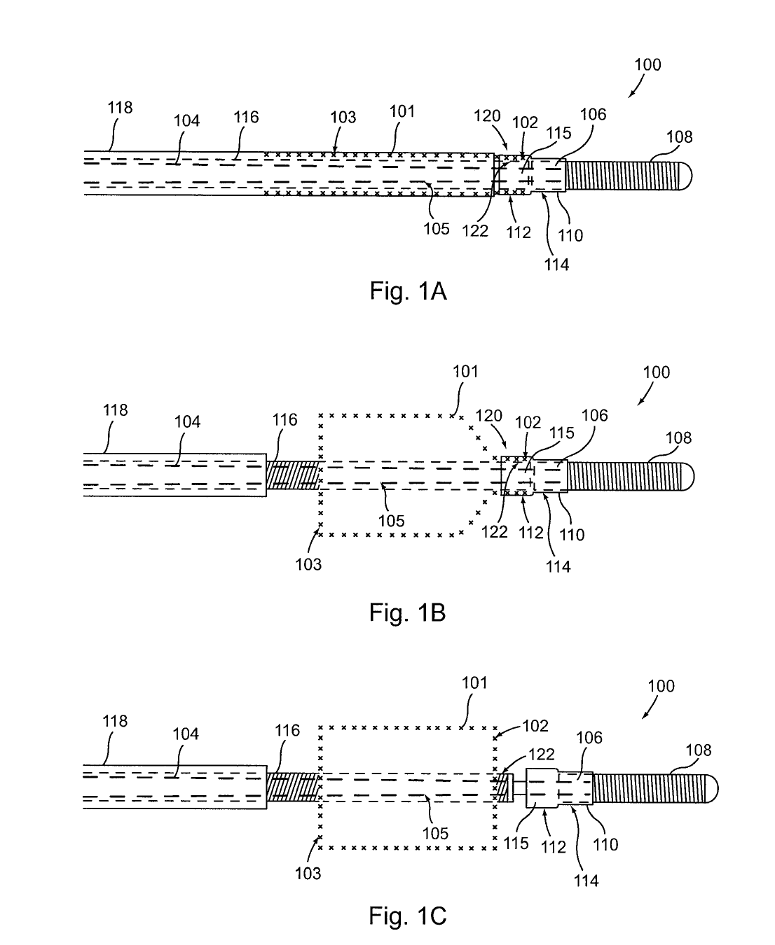

Turning to FIG. 1A, a tubular implant 101 is held in a contracted state in the implant delivery system 100. System 100 includes an elongate tubular proximal member (or outer sheath) 118. An elongate core member 104 and an elongate textured member 116 are both located within the lumen of outer sheath 118. Elongate textured member (or sleeve) 116 is configured as a tubular sleeve with the elongate core member 104, which is preferably a wire or wire-like member, slidable within the lumen of sleeve 116. Core member 104 is coupled to a hub 106 at its distal end, as well as an atraumatic tip 108, depicted here as a coiled floppy tip. Alternatively, the coil tip 108 may be omitted and core member 104 can instead be tubular (e.g., comprising hypo-tube) to allow for over-the-wire system use.

Hub 106 can be a separate body from atraumatic tip 108, in which case core member 104 can be glued, soldered, welded, clamped or otherwise fastened thereto. Alternatively, with coil tip 108, hub 106 can be formed by directly gluing, soldering or welding core member 104 to floppy tip 108 such that a proximal portion of coil tip 108 is transformed into a rigid body that acts as the hub. Core member 104 can have a tapered portion 105 adjacent hub 106 to improve distal flexibility of the delivery system.

A tubular mini-sheath 110 is coupled about hub 106 at the distal end of core member 104. This distal mini-sheath 110 includes a proximal section 112 having a relatively larger diameter than the adjacent distal section 114, which is fastened about hub 106. Proximal section 112 of distal mini-sheath 110 defines a recess or lumen 115 that can house distal end portion 102 of implant 101 and sleeve 116. Proximal section 112 can thus act as the distal housing. One of ordinary skill in the art will readily recognize that other restraints or non-tubular housings can likewise be used.

In this embodiment, textured sleeve 116 is a multi-filar coil or tube and is used to create a frictional lock with implant 101, which is a braided implant. Example medical grade multi-filar elements can include HELICAL HOLLOW STRAND (HHS) cable offered by FORT WAYNE METALS of Fort Wayne, Ind. and ACTONE offered by ASAHI INTECC CO., LTD. of Japan.

Distal end portion 102 of implant 101 is held in contact with the textured surface at the distal end portion of multi-filar sleeve 116 by distal housing 112. This contacting surface of multi-filar sleeve 116 is distal implant interface 122. Distal housing 112 closely fits over implant 101 to maintain implant end portion 102 in a state of frictional lock with the distal end portion of sleeve 116. It can be so-set by heat shrinking and/or necking down the tubing.

Sleeve 116 has sufficient resiliency to retain its shape and resist any inward pressure from distal housing 112. The combination of housing 112 and the distal end portion of sleeve 116 form distal friction-release latch 120 for the distal end portion 102 of implant 101. The friction-release latch can also be referred to as a retainer, securement or lock.

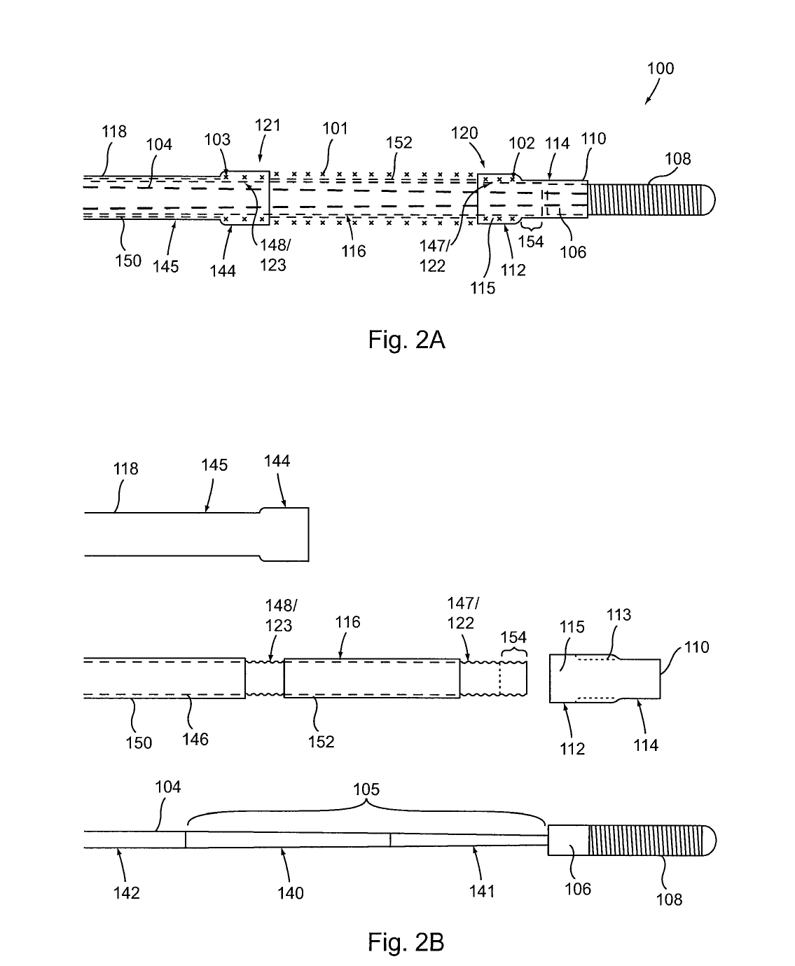

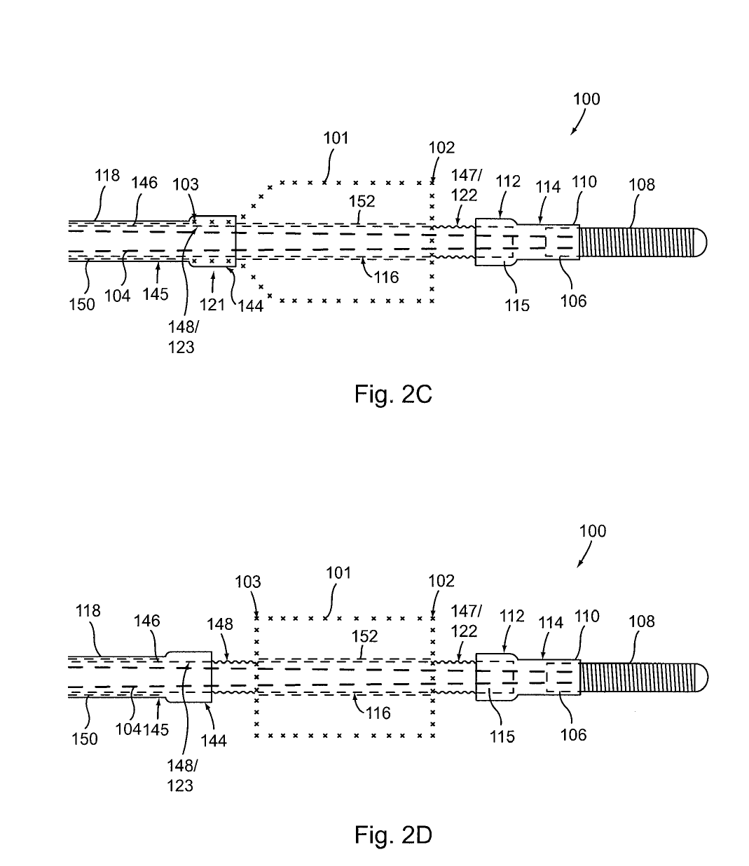

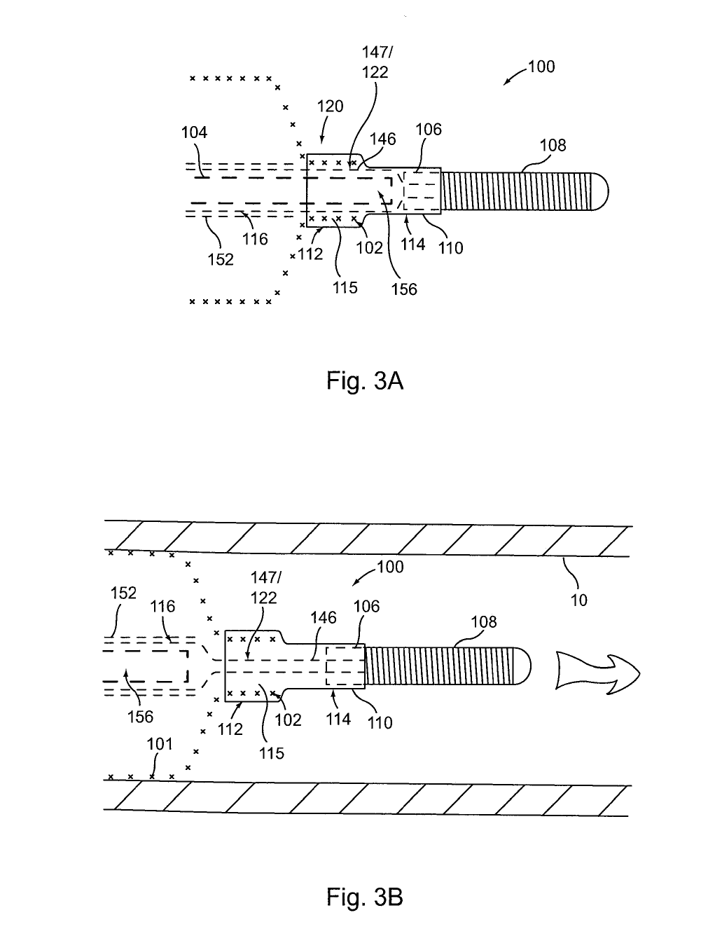

The distal end of outer sheath 118 is in close proximity to the proximal end of mini-sheath 110 and covers substantially the entire remaining portion of implant 101. Because the textured multi-filar sleeve 116 extends proximally along the length of implant 101, the entire length of implant 101 within outer sheath 118 may optionally also be held in a state of frictional lock. If so configured, such as in FIGS. 2A-2D, outer sheath 118 can act as the proximal housing and the contacting surface of multi-filar sleeve 116 is referred to as proximal implant interface 122. The combination of the proximal housing and multi-filar sleeve 116 form proximal friction-release latch 121 for the proximal end portion 103 of implant 101. It should be noted, however, that because outer sheath 118 covers the majority of implant 101 and thereby retains implant 101 in its contracted state, the formation of frictional lock 121 is not necessary and can be omitted.

Namely, when a sheath 118 substantially covering the implant is provided, it may be slightly oversized so that it is not forced into contact with sleeve 116. Under such conditions (as illustrated in FIGS. 1A-C) sheath 118 is easily withdrawn due to 1) reduced frictional forces and/or 2) stretching and reduced radial expansion force of the implant 101 caused by proximal retraction of sheath 118 during withdrawal.

FIG. 1B depicts system 100 after outer sheath 118 has been proximally retracted to expose proximal end portion 103 of implant 101. Implant 101, in this embodiment, is self-biased to expand. (i.e., self-expanding). Once exposed, proximal end portion 103 of implant 101 is free to expand to an expanded state as depicted here. Lesser expansion may be observed when deployed in a lumen. Other expansion devices can be used to transition (in the case of no implant self-bias) or facilitate the transition of, implant 101 to the expanded state. Here, only proximal end portion 103 has expanded. Distal end portion 102 is still retained within distal housing 112 by distal latch 120. This manner of deployment allows a controlled release of the implant. For instance, the medical professional is free to image the location and deployment of implant 101 before full release. It may be repositioned more distally. The implant can also be fully retrieved by pulling it with the whole delivery system back any larger catheter (typically a guiding) used for support in navigating to a site for deployment.

As expanded, the implant has foreshortened to shown the texture of cable 116. This texture, alone, is advantageously used for the distal latch/lock. And may be used to lock the implant along the length of the sleeve as well. So-configured, a minimal number of layers of material is employed, while still achieving controlled function.

Of course, an intermediate lubricious polymer liner (e.g., PTFE) can be interposed between the sleeve and core member. Alternatively, the core member may be so-coated and/or impregnated.

In any case, FIG. 1C depicts implant 101 after full deployment. Elongate core member 104 has been distally advanced with respect to sleeve 116. This action has advanced distal housing 112 from distal end portion 102 of implant 101, thereby releasing latch 120 and allowing distal end portion 102 to expand. At this point, delivery is complete and system 100 can be withdrawn through implant 101 and out of the patient's body. System 100 can be withdrawn in the state shown in FIG. 1C or can be collapsed towards the configuration of either FIG. 1A or FIG. 1B first.

FIG. 2A depicts another example embodiment of implant delivery system 100 in a state suitable for advancement through the patient's vasculature. FIG. 2B is an assembly view depicting the various components of system 100 described with respect to FIG. 2A. Like the previous embodiment, implant delivery system 100 includes core member 104, elongate sleeve 116, distal mini-sheath 110 and outer sheath 118. Here, sheath 118 includes a distal section 144 that defines the proximal housing and has a relatively larger diameter than the adjacent proximal section 145. Textured sleeve 116 includes a braided shaft 146, an intermediate covering (or jacket) 152 and an optional proximal covering (or braid jacket) 150.

Braid jackets 150 and 152 are preferably fixed to braided shaft 146 and can be formed in numerous ways. By non-exhaustive example, jackets 150 and 152 can be formed by applying heat shrink tubing or by extruding the jacket material onto braided shaft 146 and then removing or stripping the extrusion from the desired portions of braided shaft 146 (e.g., by laser ablation). Those portions can include a distal exposed braid portion 147 and a proximal exposed braid portion 148, each of which extend about the entire periphery of sleeve 116.

Typically, jacket 152 performs a structural function as further described below. Jacket 150 may be so-constructed as well. In which case, it serves as the primary catheter shaft of the device, providing pushability and torquabilty. However, layer 150 it may instead be a non fixed/floating polymeric liner. As a intermediate liner layer (e.g., comprising PTFE), it may simply provide an improved lubricous interface for sheath 118 removal. Still further, jacket 150 may comprise a multi-layer structure (e.g., as comprising a PTFE floating liner set over a heat-shrink PET jacket gripping the braid) to serve both functions.

Core member 104 may have a generally constant diameter section 142 along the length of the element. Tapered portion 105, which is distal to constant diameter section 142, can itself include one or more tapered sections for enhanced flexibility as noted above. Here, a first tapered section 140 is located adjacent section 142 and is followed by a distal tapered section 141 which tapers to a successively greater extent. Implant 101 is shown in FIG. 2A in its contracted state with end portions 102 and 103 retained within latches 120 and 121, respectively. Exposed braid portions 147 and 148 are positioned corresponding to the end portions 102 and 103, respectively, of implant 101. Here, exposed braid portions 147 and 148 are distal and proximal implant interfaces 122 and 123, respectively. Intermediate braid jacket 152 likewise corresponds to the intermediate section of implant 101 between distal housing 112 and proximal housing 144.

Intermediate braid jacket 152 stabilizes and supports braided shaft 146 in resistance to compressive force applied by implant 101 between the distal and proximal interfaces. As mentioned herein, implant 101 is held in a stretched or lengthened state where the radial dimension of the implant is decreased. This decreased radial dimension, or further stretching of implant 101 (as could occur during release), is resisted by the underlying sleeve 116. The implant can also apply a compressive force that tends to pull the distal and proximal interfaces towards each other. Unconstrained, this compression could cause the portions of braided shaft 146 having interfaces 122 and 123 to likewise compress and expand in diameter, thereby negatively effecting the crossing profile of the delivery system. The presence of intermediate braid jacket 152 resists this radial expansion and prevents compression of shaft along its longitudinal axis. Accordingly, jacket 152 is preferably a non-expandable constraint capable of preventing the underlying braid shaft 146 from expanding, as well as the adjacent interface sections 122/123. Shaft 146 may be braided at a diameter larger than as constrained by the jacket and/or mini-sheaths. As a result, the interface sections 122/123 may bulge or stand outward to offer improved anchoring/locking with the implant. However, at least the end of braided shaft 146 is preferably compressed and heat treated in the configuration depicted here so as to retain that shape. Exposure of the distal end of braided shaft 146, such as at exposed braided section 147, will thus not result in expansion of the unconstrained braid towards the relaxed diameter. Absent this heat treatment, or other restraining means, distal exposed braid section 147 would flare outwards from intermediate jacket 152 if fully exposed.

To accommodate an untreated braided shaft 146, the reduced diameter section 114 of distal housing 110 can be extended relative to hub 106. This extended section, depicted with dotted line 113 in FIG. 2B, overlaps the distal-most portion 154 of braided shaft 146 and prevents shaft 146 from expanding or flaring outwards even when the implant is released.

FIGS. 2C-D are side views depicting the example embodiment described with respect to FIGS. 2A-B during various stages of implant deployment. FIG. 2C depicts system 100 after release of distal end portion 102 of implant 101. To accomplish release, distal housing 112 is advanced distally by advancing core member 104 similar to that described previously. Implant 101 is now in a partially deployed state and the medical professional can again image and/or repositioned implant 101 as desired, but this time with the proximal end of the implant constrained Release of proximal end portion 103 of implant 101 is depicted in FIG. 2D. This is accomplished by proximally retracting sheath 118. At this point, implant 101 is fully deployed and delivery system 100 can be withdrawn in the configuration depicted here or after collapsing back to the configuration of FIG. 2C (without implant 101).

FIGS. 3A-B views depicting another example embodiment of system 100 during various stages of deployment within the body's vasculature. Referring first to FIG. 3A, core member 104 includes a distal portion 156 which is used to hold-open/wedge braided shaft 146 against implant 101 within a distal housing 112, thereby defining distal lock 120. Wedge-like portion 156 can be a rigid member attached to (or formed on) the wire-like core member. Alternatively, wedge-like portion can simply be the distal portion of core member 104, configured to act as a wedge. In this region, core member 104 is not otherwise connected to distal sheath 110 or hub 106. Instead, the distal end of braided shaft 146 is coupled directly to hub 106 about which distal sheath 110 is fixed.

In each of FIGS. 4A-B, proximal end portion 103 of implant 101 has been released in a manner similar to that described with respect to FIG. 1B or FIG. 2D. FIG. 3B depicts core member 104 after it has been proximally withdrawn from distal housing 112. Withdrawal of core member 104 and wedging portion 156 allows braided shaft 146 to collapse to a relatively more narrow diameter as depicted here. (Preferably, in this embodiment, braided shaft 146 is heat treated in such a reduced diameter configuration to allow for such action.) Core member withdrawal releases latch 120 and allows implant 101 to be fully delivered by advancing the entire system 100 distally as shown by the arrow of FIG. 3B. In one mode of delivery, implant 101 is retained in position by friction with the vessel wall 10 and allows system 100 to be advanced with respect to implant end portion 102 as shown. As system 100 is advanced, end portion 102 is freed and allowed to expand to the expanded state. After which, the delivery device is withdrawn.

It should be noted that various embodiments are described herein with reference to one or more numerical values. These numerical value(s) are intended as examples only and in no way should be construed as limiting the subject matter recited in any apparatus or method claim, absent express recitation of a numerical value in that claim.

The systems, devices and methods described herein are done so with regard to example vascular applications, but are not limited to such. When used in one example vascular application, the implant preferably has an expanded length of between about 10 mm and 50 mm, more preferably, between about 10 mm and 30 mm. The implant preferably has an expanded diameter of between about 2 mm and 8 mm, more preferably, between about 2.5 mm and 5.5 mm. The implant typically lengthens by between about 25% and 50% when transitioned to the contracted state. The length of the interfaces, or contacting surfaces, on the textured elongate member (sleeve 116) are preferably between about 0.5 mm and 5 mm, more preferably, between about 2 mm and 3 mm. The types of braid used for the implant can vary widely. In one example, the braid includes between about 24 and 96 wires/ends and, more preferably, between about 48 and 64 wires. The wire size is preferably between about 0.0008 inch (8 ten-thousandths) and 0.0025 inch, more preferably, between about 0.0015 and 0.002 inch. Uniform wire thickness or a combination of wire thicknesses may be braided together. The system is preferably configured with a crossing profile suitable for a commercially available microcatheter, typically between 0.0021 inch and 0.0027 inch, but up to 0.039 inch. The system can also be used with much larger catheters, such as a 4 french guide catheter.

Radiopacity may be inherent to the braid material (e.g., as when the stent comprises Stainless Steel, CoCr or platinum-containing drawn-filled Nitinol tubing). Or separate members (e.g., platinum wire) may be woven into the implant. Still further, platinum marker coils may be crimped, interwoven or soldered within the braid matrix.

While the embodiments are susceptible to various modifications and alternative forms, specific examples thereof have been shown in the drawings and are herein described in detail. It should be understood, however, that these embodiments are not to be limited to the particular form disclosed, but to the contrary, these embodiments are to cover all modifications, equivalents, and alternatives falling within the spirit of the disclosure. Furthermore, to the extent multiple equivalent species are described herein, recitation of an individual species in the recited claims should not be interpreted as a donation of the subject matter of the unrecited species to the public. Also, to the extent equivalent species are not recited herein, this should not be interpreted as an express or implied admission that said unrecited species are not in fact equivalents, or that said unrecited species would not be obvious to one of ordinary skill in the art after reading this disclosure.

The inventive subject matter includes the methods set forth herein in terms of method of manufacture, preparation and/or use. The methods may be performed using the subject devices and sometimes by other means. The methods may include the act of providing a suitable device. Such provision may be performed by the end user. In other words, the act of “providing” merely requires that the end user access, approach, position, set-up, grasp or otherwise obtain the requisite device for the subject method. Methods recited herein may be carried out in any order of the recited events which is logically possible, as well as in the recited order of events.

Though the subject matter described herein has been done so in reference to several examples, optionally incorporating various features, the inventive subject matter is not to be limited to that which is described or indicated as contemplated with respect to each embodiment. Also, it is contemplated that any optional feature of the inventive variations described may be set forth and claimed independently, or in combination with any one or more of the features described herein.

Reference to a singular item, includes the possibility that there are plural of the same items present. More specifically, as used herein and in the appended claims, the singular forms “a,” “an,” “said,” and “the” include plural referents unless specifically stated otherwise. In other words, use of the articles allow for “at least one” of the subject item in the description above as well as the claims below. It is further noted that the claims may be drafted to exclude any optional element. As such, this statement is intended to serve as antecedent basis for use of such exclusive terminology as “solely,” “only” and the like in connection with the recitation of claim elements or use of any “negative” limitation.

Without the use of such exclusive terminology, the term “comprising” in the claims shall allow for the inclusion of any additional element—irrespective of whether a given number of elements are enumerated in the claim, or the addition of a feature could be regarded as transforming the nature of an element set forth in the claims.

What is claimed is:

1. A medical delivery system adapted for the delivery of an expandable tubular implant, comprising:
an elongate textured member having a textured distal interface;
a distal housing having an open proximal end and being adapted to at least partially cover a distal end portion of a tubular implant in a contracted state, the distal end portion of the implant having a textured distal surface;
wherein the distal housing is adapted to releasably hold the textured distal surface of the implant against the textured distal interface of the elongate textured member such that the implant is frictionally locked within the distal housing until release.

2. The system of claim 1, wherein the distal housing is distally slidable with respect to the elongate textured member.

3. The system of claim 1, wherein the elongate textured member is an elongate textured tubular sleeve, the system further comprising an elongate core member slidable within the sleeve.

4. The system of claim 3, wherein the textured distal interface is formed by multi-filar cable.

5. The system of claim 3, wherein the textured distal interface is formed by braid.

6. The system of claim 3, wherein the elongate core member is coupled with the distal housing.

7. The system of claim 6, wherein the elongate core member is adapted to push the distal housing distally with respect to the elongate tubular sleeve to release the textured distal surface of the implant.

8. The system of claim 3, wherein the distal housing is a first section of a distal tubular sheath, the first section having a relatively larger diameter than a second section of the sheath located distal to the first section.

9. The system of claim 8, wherein the first and second sections of the distal tubular sheath are adapted to receive the elongate tubular sleeve.

10. The system of claim 3, wherein a distal portion of the elongate core member is configured hold the tubular sleeve in an open state at the distal textured interface.

11. The system of claim 10, wherein the elongate core member is proximally retractable to allow the sleeve to collapse at the distal textured interface to release the textured distal surface of the implant.

12. The system of claim 1, wherein the elongate textured member comprises a textured proximal interface, the system further comprising a proximal housing having an open distal end and being adapted to receive a textured proximal surface of the tubular implant in the contracted state and being adapted to releasably hold the textured proximal surface of the implant against the textured proximal interface of the elongate textured member such that the implant is frictionally locked to the elongate textured member.

13. The system of claim 12, wherein a proximal elongate tubular sheath includes the proximal housing.

14. The system of claim 13, wherein the proximal elongate tubular sheath is proximally retractable with respect to the elongate textured member to release the textured proximal surface of the implant.

15. The system of claim 12, wherein the elongate textured member comprises a non-textured surface located between the textured distal and proximal interfaces.

16. The system of claim 15, wherein the elongate textured member is a braided member and comprises a covering to the braid between the textured distal and proximal interfaces, the non-textured surface being the surface of the covering to the braid.

17. The system of claim 16, wherein the covering to the braid is a first covering, the elongate textured member comprising a second covering to the braid located along the length of the elongate textured member proximal to the textured proximal interface.

18. The system of claim 1, further comprising the implant, wherein the implant is a braided implant, the textured distal end portion of the implant being formed by the braid.

19. A medical implant delivery sub-assembly, comprising:
an elongate sleeve, in finished form and adapted for insertion into the body of a patient, comprising:
a first accessible textured surface extending about the outer periphery of the elongate sleeve;
a second accessible textured surface extending about the outer periphery of the elongate sleeve and located distal to the first textured surface; and
a jacket extending about the outer periphery of the elongate sleeve and located between the first and second textured surfaces, wherein the first and second textured surfaces are positioned to interface with an implant.

20. The medical implant delivery sub-assembly of claim 19, wherein the elongate sleeve comprises a tubular braid and the jacket comprises a polymeric sleeve configured to act as a constraint to radial expansion of the underlying tubular braid.

21. The medical implant delivery sub-assembly of claim 20, further comprising a second jacket over a substantial length of the tubular braid proximal to the first exposed textured surface.

22. A method of delivering a medical implant, comprising:
inserting an elongate braided member into the vasculature of a human patient, the elongate braided member comprising a distal textured implant interface, a proximal textured implant interface, and a non-textured surface located therebetween, wherein the distal and proximal textured interfaces are adapted for contact with distal and proximal end portions, respectively, of an expandable braided implant in a contracted state; and
advancing the elongate braided member through the vasculature to a treatment site.

23. The method of claim 22, wherein the distal textured implant interface is held in a state of frictional lock with the distal end portion of the implant by a distal housing, the method further comprising:
removing the distal housing to release the distal end portion of the implant from the state of frictional lock and allow the implant to at least partially transition to the expanded state.

24. The method of claim 23, wherein the proximal textured implant interface is held in a state of frictional lock with the proximal end portion of the implant by a proximal housing, the method further comprising:
removing the proximal housing to release the frictional lock and allow the implant to fully transition to the expanded state.

25. The method of claim 24, wherein the braided member includes a braid jacket located between the distal and proximal implant interfaces, the non-textured surface being the surface of the braid jacket.
